(19) United States
(12) Patent Application Publication
(10) Pub. No.: US 2018/0035797 A1
(43) Pub. Date: Feb. 8, 2018

(54) MULTI-FUNCTIONAL ORAL CARE DEVICE

(71) Applicant: Suresh Mahawar, Fremont, CA (US)

(72) Inventor: Suresh Mahawar, Fremont, CA (US)

(21) Appl. No.: 15/581,399

(22) Filed: Apr. 28, 2017

Related U.S. Application Data
(60) Provisional application No. 62/495,768, filed on Sep. 22, 2016.
(60) Provisional application No. 62/494,288, filed on Aug. 3, 2016.

(51) Int. Cl. A46B 15/00; A46B 5/00; A46B 9/04; A61C 15/04; A61B 17/24

(52) U.S. Cl. CPC A46B 15/0061; A61C 15/048; A61B 17/244; A46B 9/04; A46B 5/0095; A46B 2200/1066

(57) ABSTRACT

An oral care device. The oral care device includes a handle and at least one dental tool. The handle includes a substantially hollow body forming an internal compartment. The handle includes a top end and a bottom end. The dental tool may include a brush head having an attachment end. The oral care device includes a stowed position and a deployed position. The stowed position includes the dental tool disposed within the internal compartment. The deployed position includes the dental tool outside of the internal compartment with the attachment end mechanically fastened to the top end of the handle.

### CROSS-REFERENCE TO RELATED APPLICATION

This application claims the benefit of priority of U.S. provisional application No. 62/494,288, filed Aug. 3, 2016 and 62/495,768, filed Sep. 22, 2016, the contents of which are herein incorporated by reference.

### BACKGROUND OF THE INVENTION

The present invention relates to an oral care device and, more particularly, to an all in one oral care device for traveling.

While traveling, oral care devices are typically packed for an overnight trip. Oral care devices may include a toothbrush, floss, toothpaste, and a tongue cleaner. Currently, each device must be packed separately. Therefore, current oral care devices take up extra space and travelers may be prone to forgetting at least one of the oral care devices.

As can be seen, there is a need for an all in one oral care device for travelling.

### SUMMARY OF THE INVENTION

In one aspect of the present invention, an oral care device comprises: a handle comprising a substantially hollow body forming an internal compartment, the handle comprising a top end and a bottom end; and at least one dental tool comprising a brush head having a plurality of bristles and an attachment end, wherein the oral care device comprises a stowed position and a deployed position, the stowed position comprising the at least one dental tool disposed within the internal compartment and the deployed position comprising the at least one dental tool outside of the internal compartment with the attachment end mechanically fastened to the top end of the handle.

In another aspect of the present invention, a method of deploying an oral care device comprises the steps of: providing the oral care device comprising a handle comprising a substantially hollow body forming an internal compartment, the handle comprising a top end and a bottom end; and at least one dental tool comprising a brush head having a plurality of bristles and an attachment end, wherein the at least one dental tool is within the internal compartment; removing the at least one dental tool from the internal compartment; and mechanically fastening the at least one dental tool to the top end of the handle.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention;

FIG. 2 is a detail perspective view of an embodiment of the present invention;

FIG. 3 is a detail perspective view of an embodiment of the present invention;

FIG. 4 is an exploded view of an embodiment of the present invention;

FIG. 5 is an exploded view of an embodiment of the present invention; and

FIG. 6 is a section detail view of an embodiment of the present invention taken along line 6-6 in FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a multi-functional oral care device. The ergonomic design provides a better grip which allows users to control the force for oral care activities. This eliminates the chances of injury to the oral cavity, teeth and gums and significantly minimizes discomfort in hands and fingers resulting in decreased pain during use. The present invention is designed to store its components (tooth brush head, floss, and tongue cleaning cleaner) for travel. The user has all oral care components in one place including toothpaste. All of the components are protected inside the chamber off the handle which prevents damage of these components during travel. The ergonomic design of the device improves the user convenience and leads to increased frequency of oral care activities, such as tooth brushing, flossing, and tongue cleaning.

The handle of the device has a hollow cavity inside for the storage of its components (toothbrush head, and tongue cleaner, and floss). The cavity is closed in the bottom with a base cap. The threaded opening on the top of the handle allows fastening of its individual components (toothbrush head, and tongue cleaner, and floss). The fastening method could be through other mechanical mechanisms such as snap on and snap off clips and the like. The handle of the device can be cylindrical, triangular, rectangle or, multi-angular in shape. The handle can be straight, curved, or angulated. The surface of the handle may be smooth, rough, ridged, grooved, or carved. The base may be a toothpaste container. The toothpaste container is within the cavity and has its own cap on the top. The toothpaste container slides inside the cavity of the handle and fastens to the bottom end.

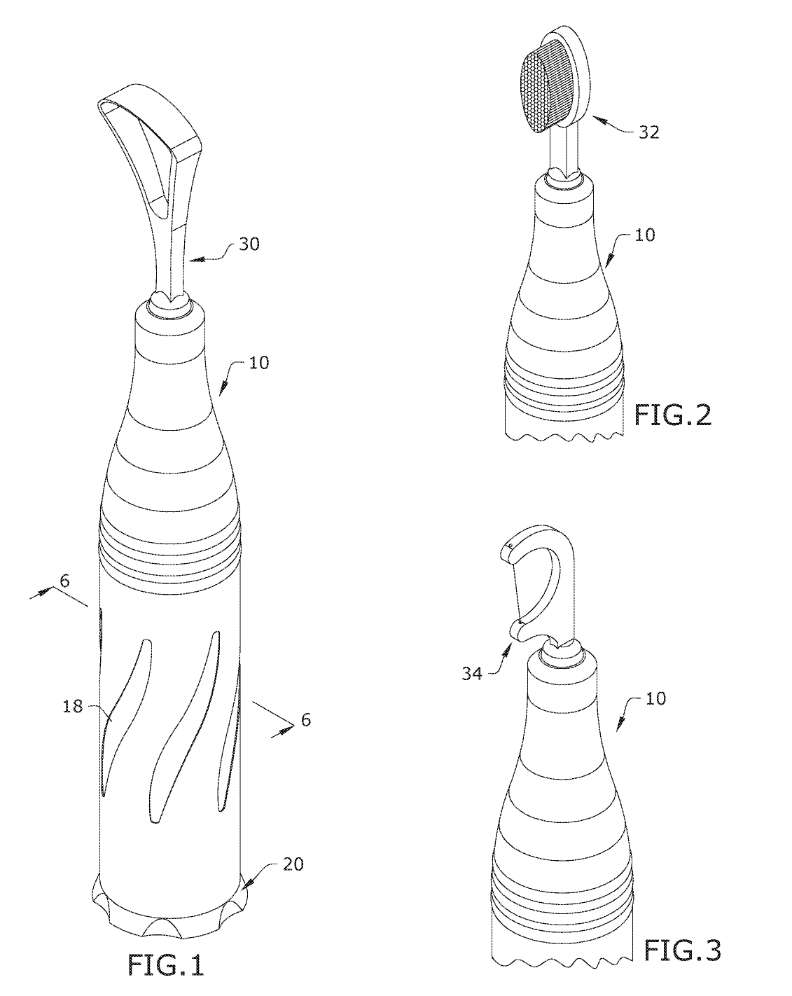

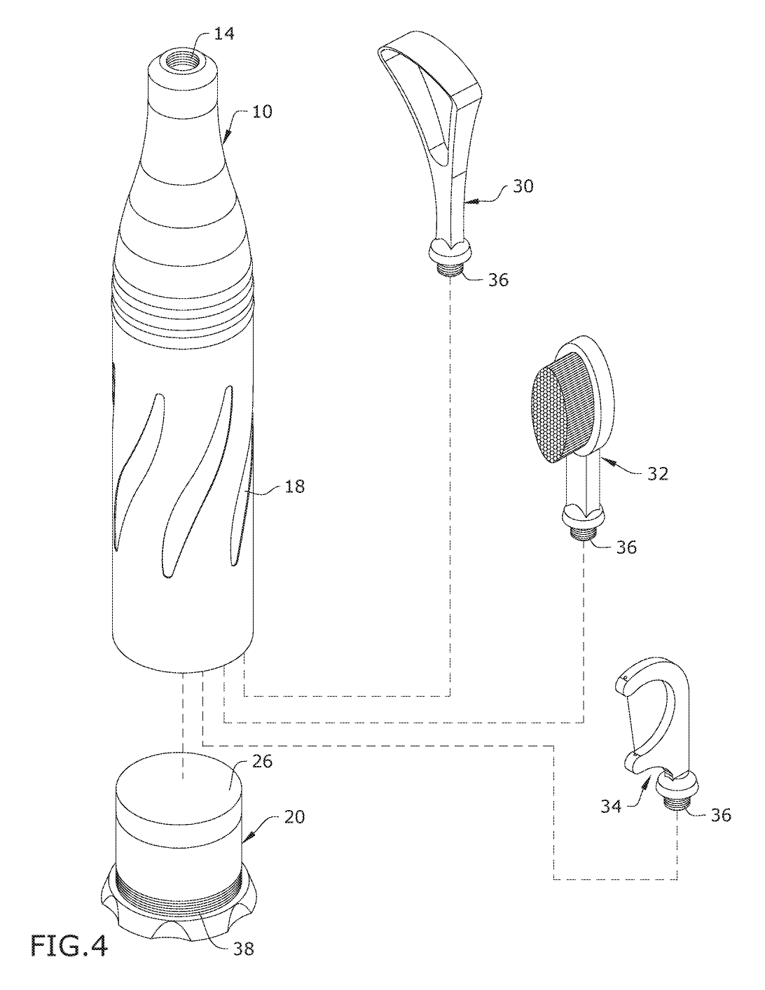

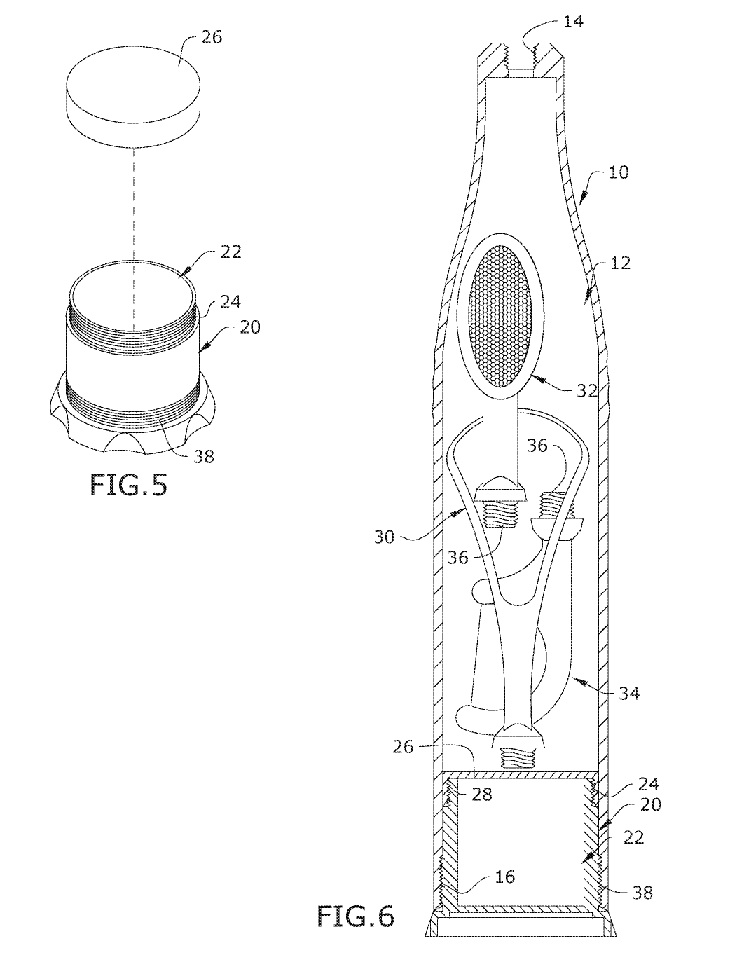

Referring to FIGS. 1 through 6, the present invention includes an oral care device. The oral care device includes a handle 10 and at least one dental tool 30, 32, 34. The handle 10 includes a substantially hollow body forming an internal compartment 12. The handle 10 includes a top end and a bottom end. The dental tool 30, 32, 34 may include a brush head 32 having an attachment end 36. The oral care device includes a stowed position and a deployed position. The stowed position includes the dental tool 30, 32, 34 disposed within the internal compartment 12. The deployed position includes the dental tool 30, 32, 34 outside of the internal compartment with the attachment end 36 mechanically fastened to the top end of the handle 10.

In certain embodiments, the handle 10 of the present invention includes a base 20 at the bottom end. The base 20 includes a base lower threaded portion 38 mechanically fastened to an internal threaded portion 16 of the internal compartment 12. The base 20 includes a substantially hollow body forming a base internal compartment 22. A cap 26 mechanically fastens to an upper rim of the base 20, thereby covering an opening leading into the base internal compartment 22. The base 20 may further include a base upper threaded portion 24 mechanically fastened to a cap threaded portion 28 of the cap 26. In such embodiments, the base internal compartment 22 may store toothpaste, mouth wash, additional floss or other dental hygiene tools.

In certain embodiments, the top end of the handle 10 includes a top threaded portion 14. The attachment end 36 of the dental tool 30, 32, 34 may be a tool threaded portion mechanically fastened to the top threaded portion 14 in the deployed position. However, the present invention is not limited to threaded connectors, and may include snap clip connectors and the like.

The present invention may include a plurality of dental tools 30, 32, 34 stowed within the handle 10 when not in use. The plurality of dental tool 30, 32, 34 may include the brush head 32, a flossing head 34 having forked arms and a flossing head attachment end 36, and a tongue cleaning head 30 having a tongue cleaning portion and a tongue cleaning attachment end 36. The user may further floss their teeth by mechanically fastening the flossing head 34 to the top end. The user may then brush their teeth by replacing the flossing head 34 with the brush head 32. The user may then clean their tongue by replacing the brush head 32 with the tongue cleaning head 30. Once the user is finished, the user may clean each of the dental tools 30, 32, 34, allow the dental tools 30, 32, 34 to dry, and place the dental tools 30, 32, 34 back into the handle 10.

The handle 10 of the present invention may be ergonomically shaped so that users may easily grasp the handle 10. For example, the handle 10 may include a body having a larger diameter and a neck extending from the body that tapers to the top end which has a smaller diameter. The enlarged body allows user's to easily grasp and manipulate the tools. Further, the enlarged body provides for a larger internal compartment 12 to store different dental tools 30, 32, 34. The handle 10 may further include a rubberized gripping 18 on the outer surface so the user may have an increased grip while using the present invention.

The present invention may further include a method of deploying an oral care device. The method includes the steps of: providing the oral care device mentioned above; unscrewing the base, thereby uncovering an opening formed at the bottom end leading into the internal compartment; removing the at least one dental tool from the internal compartment; and screwing the tool threaded portion to the top threaded portion, thereby mechanically fastening the at least one dental tool to the top end of the handle. As mentioned above, the plurality of dental tools may include the brush head, the flossing head, and the tongue cleaning head. The user may further floss their teeth by mechanically fastening the flossing head to the top end. The user may then brush their teeth by replacing the flossing head with the brush head. In certain embodiments, the user may open the cap of the base and use toothpaste disposed within the base. The user may then clean their tongue by replacing the brush head with the tongue cleaning head. Once the user is finished, the user may clean each of the dental tools, allow the dental tools to dry, and place the dental tools back into the handle.

The components of the device (handle, toothbrush head, and tongue cleaner, and floss) may be made of plastic or similar material for durability and strength. The components may be may by molding or by 3-dimensional printing. The floss threads may be made of nylon or other material. The brush of the toothbrush head may be made of nylon or other material.

It should be understood, of course, that the foregoing relates to exemplary embodiments of the invention and that modifications may be made without departing from the spirit and scope of the invention as set forth in the following claims.

What is claimed is:

1. An oral care device comprising:
a handle comprising a substantially hollow body forming an internal compartment, the handle comprising a top end and a bottom end; and
at least one dental tool comprising a brush head having a plurality of bristles and an attachment end, wherein
the oral care device comprises a stowed position and a deployed position, the stowed position comprising the at least one dental tool disposed within the internal compartment and the deployed position comprising the at least one dental tool outside of the internal compartment with the attachment end mechanically fastened to the top end of the handle.

2. The oral care device of claim 1, wherein the handle further comprises a base at the bottom end comprising a base lower threaded portion mechanically fastened to an internal threaded portion of the internal compartment.

3. The oral care device of claim 2, wherein the base comprises a substantially hollow body forming a base internal compartment, wherein a cap mechanically fastens to an upper rim of the base, thereby covering an opening leading into the base internal compartment.

4. The oral care device of claim 3, wherein the base comprises a base upper threaded portion mechanically fastened to a cap threaded portion of the cap.

5. The oral care device of claim 1, wherein the top end comprises a top threaded portion, and the attachment end of the at least one dental tool is a tool threaded portion mechanically fastened to the top threaded portion in the deployed position.

6. The oral care device of claim 1, wherein the at least one dental tool comprises a plurality of dental tools.

7. The oral care device of claim 6, wherein the plurality of dental tool comprises the brush head, a flossing head comprising forked arms and a flossing head attachment end, and a tongue cleaning head comprising a tongue cleaning portion and a tongue cleaning attachment end.

8. The oral care device of claim 1, further comprising a rubberized gripping disposed on the outer surface of the handle.

9. The oral care device of claim 1, wherein the handle comprises a body comprising a larger diameter and a neck extending from the body that tapers to the top end comprising a smaller diameter.

10. A method of deploying an oral care device comprising the steps of:
providing the oral care device comprising a handle comprising a substantially hollow body forming an internal compartment, the handle comprising a top end and a bottom end; and at least one dental tool comprising a brush head having a plurality of bristles and an attachment end, wherein the at least one dental tool is within the internal compartment;
removing the at least one dental tool from the internal compartment; and
mechanically fastening the at least one dental tool to the top end of the handle.

11. The method of claim 10, wherein the handle further comprises a base at the bottom end comprising a base lower threaded portion mechanically fastened to an internal threaded portion of the internal compartment.

12. The method of claim 11, wherein the step of removing the at least one dental tool from the internal compartment further comprises the step of:
unscrewing the base, thereby uncovering an opening formed at the bottom end leading into the internal compartment.

13. The method of claim 10, wherein the top end comprises a top threaded portion and the attachment end of the at least one dental tool is a tool threaded portion.

14. The method of claim 13 wherein the step of mechanically fastening the at least one dental tool to the top end of the handle comprises the step of:
screwing the tool threaded portion to the top threaded portion.

15. The method of claim 10, wherein the at least one dental tool comprises a plurality of dental tools.

16. The method of claim 15, wherein the plurality of dental tool comprises the brush head, a flossing head comprising forked arms and a flossing head attachment end, and a tongue cleaning head comprising a tongue cleaning portion and a tongue cleaning attachment end.
